(19) United States
(12) Patent Application Publication
(10) Pub. No.: US 2010/0062534 A1
(43) Pub. Date: Mar. 11, 2010

(54) INDUCIBLE LENTIVIRAL VECTORS FOR REPROGRAMMING SOMATIC CELLS

(75) Inventors: Konrad Hochedlinger, Boston, MA (US); Nimet Maherali, Cambridge, MA (US)

Correspondence Address: DAVID S. RESNICK, NIXON PEABODY LLP, 100 SUMMER STREET, BOSTON, MA, 02110-2131, US

(73) Assignees: THE GENERAL HOSPITAL CORPORATION, Boston, MA (US); PRESIDENT AND FELLOWS OF HARVARD COLLEGE, Cambridge, MA (US)

(21) Appl. No.: 12/364,147

(22) Filed: Feb. 2, 2009

Related U.S. Application Data
(60) Provisional application No. 61/095,500, filed on Sep. 9, 2008.

(51) Int. Cl. C12N 15/86; C12N 5/00; C12N 5/08

(52) U.S. Cl. 435/456; 435/325; 435/366

(57) ABSTRACT

Described herein is a method for reprogramming a somatic cell using an inducible lentiviral vector that permits the expression of stem-cell associated genes to be turned off or on as necessary by one of skill in the art. Inducible expression of stem-cell associated genes permits the genes to be expressed until such time that induction of iPS cells occurs and then expression can be turned off to prevent the pathological growth of cells leading to e.g., cancer or teratoma. Also described herein are secondary cell compositions, the use of which can speed the production of iPS cells, providing for a faster, more efficient system for iPS cell induction.

### CROSS REFERENCE TO RELATED APPLICATION

This application claims benefit under 35 U.S.C. §119(e) of the U.S. Provisional Application No. 61/095,500 filed on Sep. 9, 2008, the contents of which are incorporated herein by reference in their entirety.

### GOVERNMENT SUPPORT

This invention was made with Government support under Grant No. DP20D003266-0 awarded by the National Institutes of Health. The Government has certain rights in the invention.

### FIELD OF THE INVENTION

The present invention relates to the production of induced pluripotent stem cells.

### BACKGROUND

While human fibroblasts and a multitude of mouse somatic cell types can be reprogrammed to pluripotency by ectopic expression of transcription factors (Takahashi and Yamanaka, 2006; Maherali et al, 2007; Okita et al, 2007; Wernig et al. 2007; Takahashi et al, 2007; Yu et al, 2007; Lowry et al, 2007; Aoi et al, 2008; Hanna et al, 2008; Stadtfeld et al, 2008a), the conversion is highly inefficient (˜0.01%), making it difficult to examine the underlying molecular events.

### SUMMARY

Induced pluripotent stem cells are a type of pluripotent stem cell artificially derived from a somatic cell by providing for the expression of stem cell-associated genes. iPS cells are typically derived by viral delivery of stem cell-associated genes into adult somatic cells (e.g., fibroblasts). Typically the production of iPS cells has been performed by introducing a nucleic acid sequence using a genome-integrating vector (e.g., retroviral vector or lentiviral vector). Described herein is a method for reprogramming a cell using an inducible lentiviral vector that permits the expression of stem-cell associated genes to be turned off or on by one of skill in the art. Inducible expression of stem-cell associated genes permits the genes to be expressed until such time that induction of iPS cells occurs and then expression can be turned off to prevent the pathological growth of cells leading to e.g., cancer or teratoma.

One aspect described herein is a method for producing an induced pluripotent stem cell from a somatic cell, the method comprising: (a) contacting a somatic cell with an inducible lentiviral vector comprising a nucleic acid sequence encoding at least one reprogramming factor under the control of an inducible expression control element; (b) placing a somatic cell of step (a) under conditions that induce expression via said inducible expression control element; and (c) isolating a reprogrammed cell of step (b).

In one embodiment of this aspect and all other aspects described herein, step (b) comprises contacting said somatic cell with an effective amount of a regulatory agent that controls expression form the inducible expression control element.

In another embodiment of this aspect and all other aspects described herein, the inducible expression control element is a tetracycline responsive element.

In another embodiment of this aspect and all other aspects described herein, step (b) comprises contacting the somatic cell of step (a) with an effective amount of doxycycline.

In one embodiment of this aspect and all other aspects described herein, withdrawal of the effective amount of the regulatory agent permits reversible differentiation of the reprogrammed cell.

In another embodiment of this aspect and all other aspects described herein, the somatic cell comprises a human cell.

In another embodiment of this aspect and all other aspects described herein, the somatic cell comprises a fibroblast.

In another embodiment of this aspect and all other aspects described herein, the somatic cell comprises a keratinocyte.

In another embodiment of this aspect and all other aspects described herein, the reprogramming factor is selected from the group consisting of Oct4, Sox2, c-Myc and Klf4.

In another embodiment of this aspect and all other aspects described herein, the reprogramming factor includes each of Oct4, Sox2, c-Myc, and Klf4.

In another embodiment of this aspect and all other aspects described herein, the reprogramming factors further comprise NANOG.

In another embodiment of this aspect and all other aspects described herein, the production of the induced pluripotent stem cell is evidenced by detection of a stem cell marker.

In another embodiment of this aspect and all other aspects described herein, the stem cell marker is selected from the group consisting of SSEA1, CD9, Nanog, Fbx15, Ecat1, Esg1, Eras, Gdf3, Fgf4, Cripto, Dax1, Zpf296, S1c2a3, Rex1, Utf1, Oct4, SOX2, and Nat1.

Also described herein is a secondary cell composition comprising a cell having a nucleic acid encoding at least one reprogramming factor operatively linked to an inducible promoter, wherein said at least one reprogramming factor is not substantially expressed.

In one embodiment of this aspect and all other aspects described herein, the cell comprises a fibroblast phenotype.

In another embodiment of this aspect and all other aspects described herein, induction of expression of at least one reprogramming factor initiates cellular reprogramming to an iPS cell phenotype.

In another embodiment of this aspect and all other aspects described herein, the cell comprises a human cell.

In another embodiment of this aspect and all other aspects described herein, the reprogramming factor is selected from the group consisting of Oct4, Sox2, c-Myc, NANOG, and Klf4.

In another embodiment of this aspect and all other aspects described herein, the reprogramming factor includes each of Oct4, Sox2, c-Myc, and Klf4.

In another embodiment of this aspect and all other aspects described herein, the reprogramming factor further comprises NANOG.

In another embodiment of this aspect and all other aspects described herein, the cell is propagated in culture.

In another embodiment of this aspect and all other aspects described herein, the reprogramming factor operatively linked to inducible promoter is integrated into the genome of the cell. In another embodiment of this aspect and all other aspects described herein, a plurality of copies of an integrated reprogramming factor is present in each cell. For example, at least 2 copies of each integrated reprogramming factors, preferably at least 3, at least 4, at least 5, at least 6, at least 7, at least 8, at least 9, at least 10, at least 20, at least 30, at least 40, at least 50, at least 60, at least 70, at least 80, at least 90, at least 100, at least 250, at least 500, at least 1000 copies or more are present in each cell.

### DEFINITIONS

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by the ability to differentiate to more than one cell type, preferably to all three germ layers, as assayed using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers.

The term “re-programming” as used herein refers to the process of altering the differentiated state of a terminally-differentiated somatic cell to a pluripotent phenotype. A “re-programming factor” as that term is used herein refers to any factor or combination of factors that promotes the re-programming of a somatic cell and can include, for example at least one nucleic acid sequence encoding a transcription factor (e.g., c-Myc, Oct4, Sox2 and/or Klf4).

By “differentiated primary cell” or “somatic cell” is meant any primary cell that is not, in its native form, pluripotent as that term is defined herein. It should be noted that placing many primary cells in culture can lead to some loss of fully differentiated characteristics. However, simply culturing such cells does not, on its own, render them pluripotent. The transition to pluripotency requires a re-programming stimulus beyond the stimuli that lead to partial loss of differentiated character in culture. Re-programmed pluripotent cells (also referred to herein as “induced pluripotent stem cells”) are also characterized by the capacity for extended passaging without loss of growth potential, relative to primary cell parents, which generally have capacity for only a limited number of divisions in culture.

The term “vector” refers to a carrier DNA molecule into which a DNA sequence can be inserted for introduction into a host cell. An “expression vector” is a specialized vector that contains the necessary regulatory regions needed for expression of a gene of interest in a host cell. In some embodiments the gene of interest is operably linked to another sequence in the vector. It is preferred that the viral vectors used herein are replication defective, which can be achieved for example by removing all viral nucleic acids that encode for replication. A replication defective viral vector will still retain its infective properties and enters the cells in a similar manner as a replicating lentiviral vector, however once admitted to the cell a replication defective viral vector does not reproduce or multiply. The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector.

As used herein, the term “lentivirus” refers to a group (or scientific genus) of retroviruses that in nature give rise to slowly developing disease due to their ability to incorporate into a host genome. Modified lentiviral genomes are useful as viral vectors for the delivery of a nucleic acid sequence to a cell. An advantage of lentiviruses for infection of cells is the ability for sustained transgene expression. Thus, in one embodiment of the methods described herein, a lentiviral vector is employed to provide long-term expression of the transgene in a target cell.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcriptional control signals in eukaryotes comprise “promoter” and “enhancer” elements. Promoter and enhancer elements have been isolated from a variety of eukaryotic sources, including genes in yeast, insect, and mammalian cells, and viruses. Analogous control elements, i.e., promoters, are also found in prokaryotes. Such elements may vary in their strength and specificity. For example, promoters may be “constitutive” or “inducible.” A constitutive promoter is a promoter that directs expression of a gene throughout the development and life of an organism.

An “inducible promoter” is a promoter that is capable of directly or indirectly activating transcription of one or more DNA sequences or genes in response to a “regulatory agent” (e.g., doxycycline), or a “stimulus” (e.g., heat). In the absence of a “regulatory agent” or “stimulus”, the DNA sequences or genes will not be substantially transcribed. The term “not substantially transcribed” or “not substantially expressed” means that the level of transcription is at least 100-fold lower than the level of transcription observed in the presence of an appropriate stimulus or regulatory agent; preferably at least 200-fold, 300-fold, 400-fold, 500-fold or more.

As used herein, the terms “stimulus” and/or “regulatory agent” refers to a chemical agent, such as a metabolite, a small molecule, or a physiological stress directly imposed upon the organism such as cold, heat, toxins, or through the action of a pathogen or disease agent. A recombinant cell containing an inducible promoter may be exposed to a regulatory agent or stimulus by externally applying the agent or stimulus to the cell or organism by exposure to the appropriate environmental condition or the operative pathogen. Inducible promoters initiate transcription only in the presence of a regulatory agent or stimulus. Examples of inducible promoters include the tetracycline response element and promoters derived from the β-interferon gene, heat shock gene, metallothionein gene or any obtainable from steroid hormone-responsive genes.

Inducible promoters which may be used in performing the methods of the present invention include those regulated by hormones and hormone analogs such as progesterone, ecdysone and glucocorticoids as well as promoters which are regulated by tetracycline, heat shock, heavy metal ions, interferon, and lactose operon activating compounds. For review of these systems see Gingrich and Roder, 1998, Annu Rev Neurosci 21, 377-405. Tissue specific expression has been well characterized in the field of gene expression and tissue specific and inducible promoters are well known in the art. These promoters are used to regulate the expression of the foreign gene after it has been introduced into the target cell.

As used herein, the term “inducible lentiviral vector” refers to a lentiviral vector comprising e.g., a regulatory agent responsive element that permits gene transcription of a target gene to be turned on in the presence of an effective amount of a regulatory agent (e.g., doxycycline) or turned off (i.e., no gene product is produced) in the absence of an effective amount of the regulatory agent.

As used herein, the term “inducible expression control element” refers to a genetic element which controls some aspect of the expression of nucleic acid sequences and is itself controlled by a “regulatory agent”, as that term is used herein. For example, a promoter is an expression control element that facilitates the initiation of transcription of an operably linked coding region.

As used herein, the term “effective amount of a regulatory agent” refers to administration to a cell of at least the amount of a regulatory agent necessary to turn on gene transcription from an inducible lentiviral vector as measured by Western blot analysis, RT-PCR, or qRT-PCR for the gene product (i.e., mRNA or protein). It is understood that a threshold level of a regulatory agent may exist, such that below the threshold no gene transcription occurs, even if low levels of a regulatory agent are present. It is well within the abilities of one skilled in the art to test for an appropriate level of a regulatory agent in cultured cells by performing a dose-response curve and measuring e.g., mRNA production as an indicator of gene transcription. It is contemplated that the effective level of a regulatory agent can also be titrated above the minimum level needed, such that the amount of gene transcription is at an optimal, desired level for the skilled practitioner.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### BRIEF DESCRIPTION OF THE FIGURES

FIG. 1. Generation of hiPS cells using inducible lentiviruses.

1A. An exemplary experimental scheme for the generation of hiPS cells. Primary human fibroblasts and keratinocytes were infected with separate lentiviruses (LV) containing a constitutively active rtTA and doxycycline-inducible reprogramming factors. After infection, cells were seeded to feeders and doxycycline (dox) was applied for 30 days. hiPS clones were picked based on hESC-like morphology and doxycycline-independent growth.

1B. Hematoxylin and eosin stain of teratomas generated from fibroblast-derived hiPS cells. Differentiated structures from all three germ layers were present. i) Pigmented epithelium (ectoderm), ii) cartilage (mesoderm), iii) gut-like epithelium (endoderm), and iv) muscle (mesoderm).

FIG. 2. Generation of secondary hiPS cells.

2A. An exemplary experimental scheme depicting the generation of secondary hiPS cells. hiPS cells were differentiated in vitro as embryoid bodies for 7 days, then plated to adherent conditions. Fibroblast-like colonies were picked and expanded for at least three passages prior to undergoing re-induction by doxycycline.

2B. Alkaline phosphatase staining of reprogrammable cells grown in the absence or presence of doxycycline. Doxycycline was withdrawn at day 21, and colonies were stained and counted at day 30.

2C. Temporal requirement of factor expression in hiPS-derived fibroblast-like cells. 10⁴ cells were plated per time-point and doxycycline was withdrawn daily from days 4 through 19. The number of hES-like colonies that expressed Tra-1-81 were counted at day 25.

FIG. 3. Molecular characterization of hiPS cells.

3A. Quantitative RT-PCR analysis for total, endogenous, and viral gene expression of the reprogramming factors. WA09 hES cells, uninfected keratinocytes (K0) and BJ fibroblasts, and 5-factor-infected BJ fibroblasts (3 days+dox) served as controls. Values were standardized to GAPDH, then normalized to WA09 hESC (total and endogenous) or infected BJs (viral).

3B. Bisulfite sequencing of the NANOG and the OCT4 promoter regions in BJ fibroblasts, BJ fibroblast-derived hiPS, and WA09 hES cells. Promoter regions containing differentially methylated CpGs are shown. Open circles represent unmethylated CpGs; closed circles denote methylated CpGs.

3C. Temporal requirement of factor expression in keratinocytes. Cells infected with five factors were seeded at 7.5×10⁴ cells per time point and doxycycline was withdrawn every four days from days 6 through 18. The number of hES-like colonies was counted at day 30. All colonies could be expanded in the absence of doxycycline.

FIG. 4. Characterization of hiPS-derived fibroblast-like cells.

4A. Quantitative RT-PCR analysis of reprogramming factor and pluripotency gene expression in hiPS-derived fibroblasts, WA09 hES cells, and BJ fibroblasts. Values were standardized to GAPDH.

4B. Quantitative RT-PCR analysis for viral transgene reactivation in hiPS-derived fibroblast-like cells from four BJ-hiPS clones. Cells were analyzed two days after doxycycline administration. Clones #5 and #8 were generated with four factors; clones #11 and #12 with five factors. Values were standardized to GAPDH, then calculated as the ratio of expression between ±doxycycline.

FIG. 5. Visual tracking of hiPS colony formation. hiPS-derived fibroblast-like cells were induced to form secondary hiPS cells, and individual colonies were tracked during the reprogramming process. Doxycycline was withdrawn at various time points.

5A. Representative colony that failed to reprogram.

5B. Representative colony that underwent successful reprogramming.

5C. Representative colonies that gave rise to a hiPS clone; two adjacent colonies were tracked. By day 15, these colonies had regressed and an hES-like colony began to form between them (arrows), which continued to develop as hiPS cells.

Table 1. Summary of expanded hiPS cell lines.

Table 2. Primers used in this study.

### DETAILED DESCRIPTION

In order to efficiently silence expression of re-programming factors in hiPS cells to prevent cancer and/or teratoma formation, an inducible lentiviral system is used herein. A colony of hiPS cells are selected and differentiated in vitro to yield fibroblast-like cells that harbor the inducible viral transgenes required for reprogramming (referred to herein as “secondary” pluripotent cells). These cells maintain the same viral integrations that mediated the initial conversion to hiPS cells, however the proportion of cells carrying the necessary re-programming factors is increased. These fibroblast-like cells can be readily induced to form hiPS cells without further need for direct viral infection. This “secondary pluripotent cell” system produces a population of cells that can inducibly and homogeneously express the reprogramming factors with improved efficiency. In addition, the use of secondary pluripotent cells can speed the production of hiPS cells, providing for a faster, more efficient system for hiPS cell induction. Such a system provides a powerful tool for mechanistic analysis, chemical and genetic screening for factors that enhance or block reprogramming, and the optimization of hiPS cell derivation methods. In addition, the secondary cell composition provides the added advantage of being cultured in a fibroblast phenotype until an iPS cell phenotype is desired.

### Cells

While fibroblasts and keratinocytes are preferred, essentially any primary somatic cell type can be used. Some non-limiting examples of primary cells include, but are not limited to, epithelial, endothelial, neuronal, adipose, cardiac, skeletal muscle, immune cells, hepatic, splenic, lung, circulating blood cells, gastrointestinal, renal, bone marrow, keratinocytes and pancreatic cells. The cell can be a primary cell isolated from any somatic tissue including, but not limited to brain, liver, lung, gut, stomach, intestine, fat, muscle, uterus, skin, spleen, endocrine organ, bone, etc.

Where the cell is maintained under in vitro conditions, conventional tissue culture conditions and methods can be used, and are known to those of skill in the art. Isolation and culture methods for various cells are well within the abilities of one skilled in the art.

Further, the parental cell can be from any mammalian species, with non-limiting examples including a murine, bovine, simian, porcine, equine, ovine, or human. For clarity and simplicity, the description of the methods herein refers to fibroblasts as the parental cells, but it should be understood that all of the methods described herein can be readily applied to other primary parent cell types. In one embodiment, the somatic cell is derived from a human individual.

Where a fibroblast is used, the fibroblast can have flattened and irregularly shaped morphology prior to re-programming, and may not express Nanog mRNA. The starting fibroblast will preferably not express other embryonic stem cell markers. The expression of ES-cell markers can be measured, for example, by RT-PCR. Alternatively, measurement can be by, for example, immunofluorescence or other immunological detection approach that detects the presence of polypeptides that are characteristic of the ES phenotype.

### Lentiviral Vectors

Essentially any inducible lentiviral vector can be used with the methods and compositions described herein. In some embodiments, it is preferred that the lentiviral vector integrates into the host cell genome.

Lentiviral vectors useful for the methods and compositions described herein can comprise a eukaryotic promoter. The promoter can be any inducible promoter, including synthetic promoters, that can function as a promoter in a eukaryotic cell. For example, the eukaryotic promoter can be, but is not limited to, ecdysone inducible promoters, E1a inducible promoters, tetracycline inducible promoters etc., as are well known in the art.

In addition, the lentiviral vectors used herein can further comprise a selectable marker, which can comprise a promoter and a coding sequence for a selectable trait. Nucleotide sequences encoding selectable markers are well known in the art, and include those that encode gene products conferring resistance to antibiotics or anti-metabolites, or that supply an auxotrophic requirement. Examples of such sequences include, but are not limited to, those that encode thymidine kinase activity, or resistance to methotrexate, ampicillin, kanamycin, chloramphenicol, or zeocin, among many others.

### Reprogramming

The production of iPS cells is generally achieved by the introduction of nucleic acid sequences encoding stem cell-associated genes into an adult, somatic cell. In general, these nucleic acids have been introduced using retroviral vectors, and expression of the gene products results in cells that are morphologically and biochemically similar to pluripotent stem cells (e.g., embryonic stem cells). For the purposes of this application, the nucleic acid sequences are delivered using an inducible lentiviral vector. This process of altering a cell phenotype from a somatic cell phenotype to a stem cell-like phenotype is termed “reprogramming”.

Reprogramming can be achieved by introducing a combination of stem cell-associated genes including, for example Oct3/4 (Pouf51), Sox1, Sox2, Sox3, Sox 15, Sox 18, NANOG, Klf1, Klf2, Klf4, Klf5, c-Myc, 1-Myc, n-Myc and LIN28. In general, successful reprogramming is accomplished by introducing a vector encoding Oct-3/4, a member of the Sox family, a member of the Klf family, and a member of the Myc family to a somatic cell.

In one embodiment of the methods described herein, reprogramming is achieved by delivery of Oct-4, Sox2, c-Myc, and Klf4 constructs to a somatic cell (e.g., fibroblast). In one embodiment, the nucleic acid sequences of Oct-4, Sox2, c-MYC, and Klf4 are delivered using an inducible lentiviral vector.

Control of expression of re-programming factors is achieved by contacting a somatic cell having at least one re-programming factor under the control of an inducible promoter, with a regulatory agent (e.g., doxycycline) or other inducing agent. In certain inducible lentiviral vectors, contacting such a cell with a regulatory agent induces expression of the re-programming factors, while withdrawal of the regulatory agent inhibits expression. In other inducible lentiviral vectors, the opposite is true (i.e., the regulatory agent inhibits expression and removal permits expression). The term “induction of expression” refers to the administration or withdrawal of the a regulatory agent (i.e., depending on the lentiviral vector used) and permits expression of at least one reprogramming factor.

It is contemplated herein that induction of expression is only necessary for a certain portion of the re-programming process. While the time necessary for induction of expression will vary with somatic cell type used, it is generally necessary to detect at least one iPS cell in a culture prior to stopping the induction stimulus. However, it is well within the abilities of one skilled in the art to identify an appropriate time necessary to treat a somatic cell with an induction stimulus. It is contemplated herein that induction of expression may be as short as four hours, or alternatively expression of can be induced for the entire reprogramming process, as well as any integer of time in between. For example, induction of expression can be at least 4 hours, at least 5 hours, at least 6 hours, at least 12 hours, at least 24 hours, at least 48 hours, at least 3 days, at least 4 days, at least 5 days, at least 6 days, at least 7 days, at least 8 days, at least 9 days, at least 10 days, at least 11 days, at least 12 days, at least 13 days, at least 14 days, at least 2.5 weeks, at least 3 weeks, at least 3.5 weeks, at least 4 weeks, at least 5 weeks, at least 6 weeks, at least 7 weeks, at least 8 weeks, at least 3 months, or more until a desired level of induction of iPS cells is detected. It is important to note that induction of expression for long periods of time may actually be detrimental to cell viability and thus it is contemplated herein upon detection of at least one iPS cell in the culture will signal one of skill in the art to stop the induction of expression. In addition, it is further contemplated that induction of expression is stopped at least 1 day prior to using the iPS cells for stem-cell therapy, diagnostics, administration to a subject, or research purposes.

### Confirming Pluripotency and Cell Reprogramming

To confirm the induction of pluripotent stem cells, isolated clones can be tested for the expression of a stem cell marker. Such expression identifies the cells as induced pluripotent stem cells. Stem cell markers can be selected from the non-limiting group including SSEA1, CD9, Nanog, Fbx15, Ecat1, Esg1, Eras, Gdf3, Fgf4, Cripto, Dax1, Zpf296, S1c2a3, Rex1, Utf1, and Nat1. Methods for detecting the expression of such markers can include, for example, RT-PCR and immunological methods that detect the presence of the encoded polypeptides.

The pluripotent stem cell character of the isolated cells can be confirmed by any of a number of tests evaluating the expression of ES markers and the ability to differentiate to cells of each of the three germ layers. As one example, teratoma formation in nude mice can be used to evaluate the pluripotent character of the isolated clones. The cells are introduced to nude mice and histology is performed on a tumor arising from the cells. The growth of a tumor comprising cells from all three germ layers further indicates that the cells are pluripotent stem cells.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The present invention may be as defined in any one of the following numbered paragraphs.
1. A method for producing an induced pluripotent stem cell from a somatic cell, the method comprising:
   (a) contacting a somatic cell with an inducible lentiviral vector comprising a nucleic acid sequence encoding at least one reprogramming factor under the control of an inducible expression control element;
   (b) placing the somatic cell of step (a) under conditions that induce expression via inducible expression control element; and
   (c) isolating a reprogrammed cell of step (b).
2. The method of paragraph 1, wherein step (b) comprises contacting the somatic cell with an effective amount of a regulatory agent that controls expression from the inducible expression from the inducible expression control element.
3. The method of paragraph 1, wherein the inducible expression control element is a tetracycline responsive element.
4. The method of paragraph 1, wherein step (b) comprises contacting the somatic cell of step (a) with an effective amount of doxycycline.
5. The method of paragraph 4, wherein withdrawal of the effective amount of a regulatory agent permits differentiation of the reprogrammed cell.
6. The method of paragraph 1, wherein the somatic cell comprises a human cell.
7. The method of paragraph 1, wherein the somatic cell comprises a fibroblast.
8. The method of paragraph 1, wherein the somatic cell comprises a keratinocyte.
9. The method of paragraph 1, wherein the reprogramming factor is selected from the group consisting of Oct4, Sox2, c-Myc and Klf4.
10. The method of paragraph 1, wherein the reprogramming factor includes each of Oct4, Sox2, c-Myc, and Klf4.
11. The method of paragraph 10, wherein the reprogramming factor further comprises NANOG.
12. The method of paragraph 1, wherein production of the induced pluripotent stem cell is evidenced by detection of a stem cell marker.
13. The method of paragraph 12, wherein the stem cell marker is selected from the group consisting of SSEA1, CD9, Nanog, Fbx15, Ecat1, Esg1, Eras, Gdf3, Fgf4, Cripto, Dax1, Zpf296, S1c2a3, Rex1, Utf1, Oct4, SOX2, and Nat1.
14. A secondary cell composition comprising a cell having a nucleic acid encoding at least one reprogramming factor operatively linked to an inducible promoter, wherein the at least one reprogramming factor is not substantially expressed.
15. The composition of paragraph 14, wherein the cell comprises a fibroblast phenotype.
16. The composition of paragraph 14, wherein induction of expression of the at least one reprogramming factor initiates cellular reprogramming to a iPS cell phenotype.
17. The composition of paragraph 14, wherein the cell comprises a human cell.
18. The composition of paragraph 11, wherein the reprogramming factor is selected from the group consisting of Oct4, Sox2, c-Myc, NANOG, and Klf4.
19. The composition of paragraph 14, wherein the reprogramming factor includes each of Oct4, Sox2, c-Myc, and Klf4.
20. The composition of paragraph 19, wherein the reprogramming factor further comprises NANOG.
21. The composition of paragraph 15, wherein the cell is propagated in culture.

### EXAMPLES

### Example 1

### Results

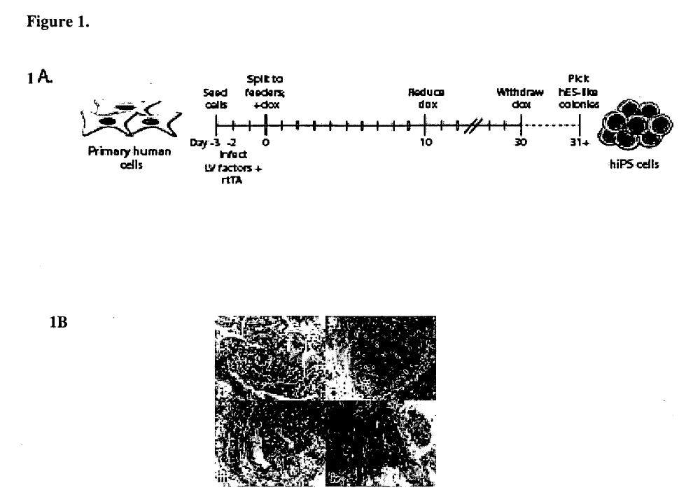

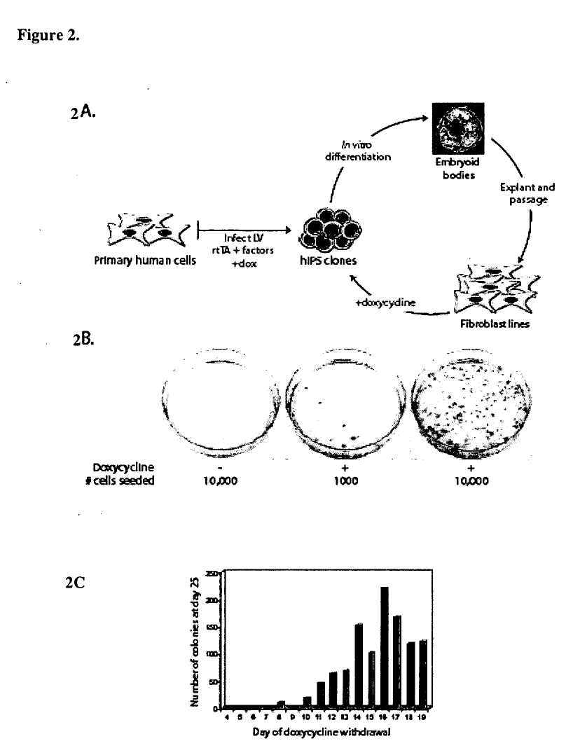

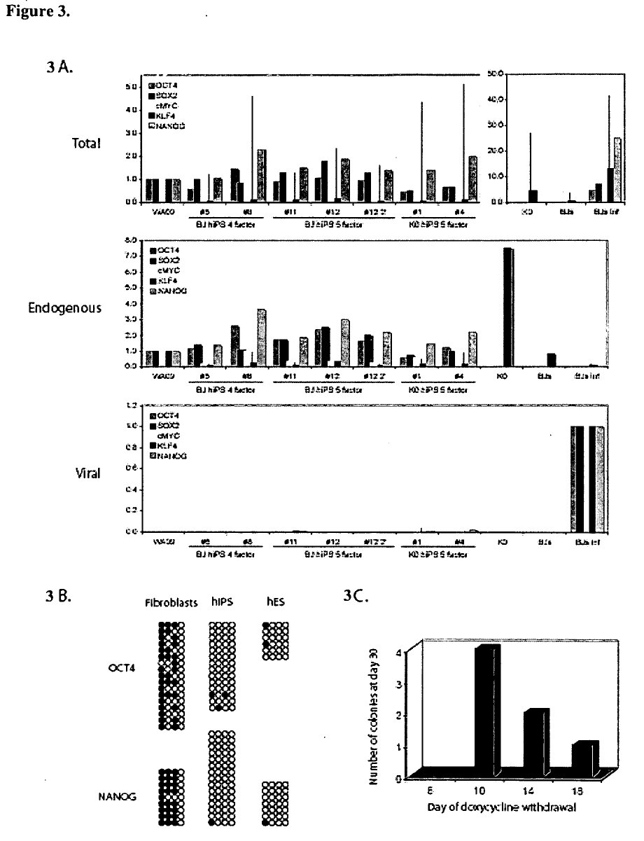

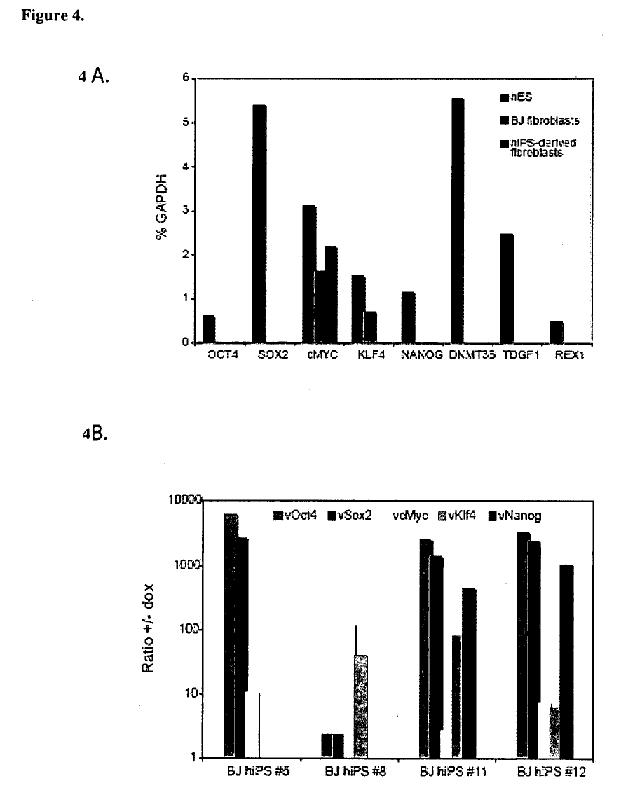

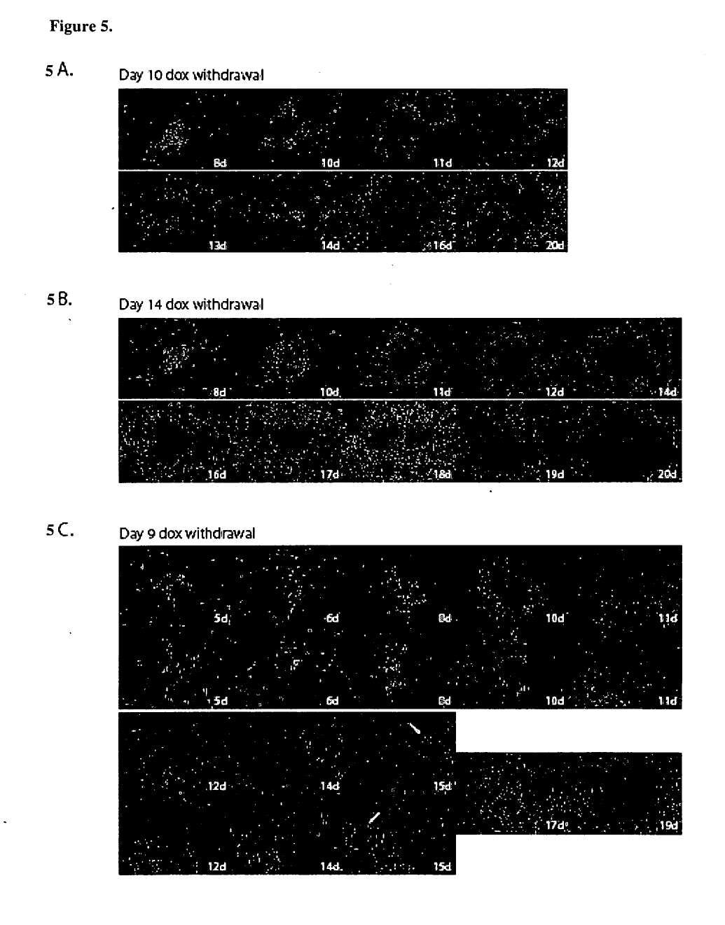

cDNAs encoding human OCT4, SOX2, cMYC, KLF4, and NANOG were cloned into doxycycline-inducible lentiviral vectors as previously described (Stadtfeld et al, 2008b). In addition, a reverse tetracycline transactivator (rtTA) driven by the ubiquitin promoter was cloned into a lentiviral vector. To generate hiPS cells, neonatal foreskin fibroblasts (BJ) and keratinocytes were infected with lentiviruses containing the rtTA and either four (OCT4, SOX2, cMYC, and KLF4; for fibroblasts only) or five reprogramming factors (4+NANOG; both fibroblasts and keratinocytes) according to the scheme in FIG. 1A. Following infection, cells were plated onto mouse embryonic fibroblast feeder cells (MEFs) under hES cell conditions and induced with doxycycline. From the fibroblast cultures, non-hES-like colonies emerged approximately two weeks after the addition of doxycycline. These colonies contained only Oct4 and Myc integrations and could not be expanded in the absence of doxycycline (data not shown). After 30 days of culture, colonies that resembled hES cells were observed, noted by a high nucleus-to-cytoplasmic ratio, prominent nucleoli, and well-defined phase-bright borders.

All hES-like colonies expressed the hES-specific surface antigen Tra-1-81 (data not shown) and could be expanded in the absence of doxycycline. Colonies that did not resemble hES cells did not express Tra-1-81 and could not be passaged independent of doxycycline. Of ˜2.5×10⁵ infected fibroblasts seeded, 4 iPS colonies were obtained from each condition (four or five factors), representing a frequency of ˜0.002%. From the keratinocyte cultures, large non-ES-like colonies appeared within one week; within three weeks, hES-like colonies appeared and could be passaged in the absence of doxycycline. Of ˜3×10⁵ cells seeded, 7 colonies emerged, similar to the frequency observed for hiPS derivation from fibroblasts (˜0.002%).

hiPS cell colonies stained positive for OCT4 protein and the hES cell-specific surface antigen Tra 1-81 (data not shown). Further, these cells showed expression of pluripotency genes from the endogenous loci and lacked expression of the viral transgenes (FIG. 3A). To assess whether hiPS cells were molecularly similar to hES cells, promoter methylation was examined and global transcriptional analysis performed. The NANOG and OCT4 promoters in fibroblast-derived hiPS cells were demethylated to a similar extent as in hES cells, in contrast to the highly methylated promoters in BJ fibroblasts (FIG. 3B), thus demonstrating epigenetic reprogramming in hiPS cells. Global analysis of gene expression in fibroblast-derived hiPS cells, hES cells, and fibroblasts was conducted by microarray through comparison of differentially-expressed genes between fibroblasts and hES cells (data not shown), indicating that hiPS cells had repressed the fibroblast program of gene expression and reactivated an embryonic program of transcription.

Pluripotency of both fibroblast-and keratinocyte-derived hiPS cells was examined in vitro through embryoid body (EB) formation. After 7 days in suspension culture, EBs were explanted and gave rise to well-defined neuronal outgrowths and beating cardiomyocyte structures (data not shown). Immunofluorescence analysis confirmed the presence of neurons (e.g., Tuj1 neuronal marker), cardiomyocytes (e.g., cardiac troponin T marker), skeletal muscle cells (e.g., MF20 skeletal muscle marker), and epithelial structures (e.g., alpha-fetoprotein epithelial and early endodermal marker) (data not shown), thus demonstrating multi-lineage differentiation. As a more stringent test of pluripotency, fibroblast-derived hiPS cells were injected either subcutaneously or under the kidney capsule of immunodeficient SCID mice to assay for teratoma formation. Tumors were recovered after 10 weeks and contained well-defined structures arising from all three embryonic germ layers (FIG. 1B), including pigmented cells (i.e. ectoderm), cartilage (i.e., mesoderm), skeletal muscle (i.e., mesoderm), and gut-like epithelium (i.e. endoderm). These results indicate that hiPS cells generated with an inducible system strongly resemble hES cells and fulfill all criteria for pluripotency.

Noting that keratinocyte-derived hiPS colonies appeared faster than fibroblast-derived hiPS cells, it was sought to determine the minimum amount of time required to convert keratinocytes to hiPS cells. To test this, keratinocytes were infected with rtTA and five factors (OCT4, SOX2, cMYC, KLF4, NANOG), and doxycycline was withdrawn at different time-points throughout the reprogramming process. The number of hES-like colonies was counted at day 30 and plotted against the day of doxycycline withdrawal (data not shown). hES-like colonies first appeared after 18 days when doxycycline had been withdrawn after 10 days. The frequency of reprogramming appeared to decline with the length of doxycycline exposure, which may reflect unfavorable culture conditions at later time points or adverse effects of continued transgene expression.

To establish the system of “secondary” hiPS cells, several fibroblast-derived hiPS clones were differentiated to fibroblast-like cells in vitro according to the scheme in FIG. 2A. hiPS cell colonies were placed in suspension culture for one week and the resulting EBs were then plated to adherent conditions. Outgrowths of fibroblast-like cells were picked and passaged a minimum of three times prior to experimental manipulation to ensure that no residual pluripotent cells were present. Quantitative RT-PCR analysis confirmed a lack of pluripotency gene expression in these populations (FIG. 4A). Alkaline phosphatase staining of reprogrammable cells grown in the absence or presence of doxycycline was performed (data not shown). Doxycycline was withdrawn at day 21, and colonies were stained and counted at day 30 (data not shown).

The ability of hiPS-derived cells to generate “secondary” hiPS cells through doxycycline addition and transfer to hiPS derivation conditions was assessed. Fibroblast-like cells derived from two hiPS clones demonstrated reprogramming in the presence, but not absence, of doxycycline (FIG. 2B). The frequency of conversion ranged from 1-3%. Re-induction of viral transgenes in hiPS-derived fibroblasts clones was also assessed by quantitative RT-PCR (FIG. 4B), demonstrating a correlation between factor reactivation and the ability of the clone to produce secondary hiPS. Clones that gave rise to secondary hiPS cells showed reactivation of all factors (BJ hiPS #11 and #12), while those that did not lacked re-expression of a factor (BJ hiPS #5) or re-expressed the factors at levels that are likely not permissive for the induction of pluripotency (BJ hiPS #8).

Secondary hiPS cells were molecularly and functionally similar to primary hiPS and hES cells. They stained positive for OCT4 and Tra-1-81 (data not shown), had a similar gene expression profile to hES cells (data not shown), and demonstrated pluripotency in vitro, generating cell types from all three embryonic germ layers (data not shown).

To determine the temporal requirement of transgene expression for secondary hiPS cell formation, 10⁴ fibroblast-like cells were seeded per time point under hiPS derivation conditions and doxycycline was withdrawn daily from days 4 through 19. The final number of Tra-1-81+hES-like colonies was counted at day 25 (FIG. 2C). hiPS colonies began to appear after withdrawal of doxycycline at day 6 (one colony), and the number of colonies increased with the time of doxycycline exposure, reaching a maximum after withdrawal at 16 days (frequency of ˜2%). This increase in frequency with length of doxycycline exposure has also been reported in the reprogramming of mouse cells (Wernig et al, 2008).

Transformed granular colonies did not appear during the reprogramming process, in contrast to the early colonies that appeared during the primary induction with direct viral infection. The lack of these background colonies coupled with the high efficiency of the secondary system enabled the inventors to monitor the progression of colonies during reprogramming. Individual colonies were tracked on a daily basis with different time points of doxycycline withdrawal (FIG. 5), and it was observed that induced cells transited through non-ES-like structures prior to acquiring a hiPS cell phenotype. Not all colonies developed fully to hiPS cells; those that did not undergo successful reprogramming began to regress two days after doxycycline withdrawal and ultimately formed fibroblast-like structures. The colonies that successfully generated hiPS cells also showed some regression after withdrawal of doxycycline, however, hES-like outgrowths gradually appeared, which could be expanded into stable hiPS cell lines.

Herein is described the use of an inducible lentiviral system for the reprogramming of human somatic cells. Using this system, neonatal foreskin fibroblasts and keratinocytes were converted to a pluripotent state that is molecularly and functionally similar to hES cells. This system also enabled us to establish the temporal requirement of factor expression for cells undergoing reprogramming. While fibroblasts relied on transgene expression for several weeks, keratinocytes required only 10 days of factor expression to revert to a state that was poised to become pluripotent. It is unknown why keratinocytes are more amenable to reprogramming. Keratinocytes, like hES cells, represent an epithelial cell type and in contrast to fibroblasts may not need to undergo a mesenchymal-epithelial transition during reprogramming (Yang and Weinberg, 2008). Differences in reprogrammability between fibroblasts and keratinocytes may also be explained by differences in the cell cycle status, epigenetic regulation, location of integrated genes, or viral infectivity. Moreover, keratinocytes express much higher levels of endogenous MYC and KLF4 than fibroblasts (FIG. 3A), which may accelerate their conversion to hiPS cells. The fast kinetics of reprogramming observed for keratinocytes indicates that these cells are useful for the development and optimization of methods to reprogram cells by transient delivery of factors.

Controlled expression of the reprogramming factors provide an inherent selection strategy by eliminating cells that are dependent upon viral transgene expression and conferring a growth advantage to fully reprogrammed cells that are doxycycline-independent. This is in contrast to the constitutive retro-and lentiviral systems that have so far been used to reprogram human cells, where the viral transgenes maintain expression in the hiPS cell state. The persistence of viral gene expression could have deleterious effects in applications such as in vitro disease modeling; for example, the overexpression of Oct4 or Sox2 has been shown to promote the differentiation of mouse ES cells (Niwa et al, 2000; Kopp et al, 2008), indicating that their continued expression during in vitro differentiation of hiPS cells may bias the resulting cell fate.

Due to the low efficiency of reprogramming, a system of “secondary” pluripotent cells was used herein, which permits hiPS-derived differentiated cells to be developed at a high frequency. The >100-fold increase observed was likely attributed to the ability to reactivate all factors within a given cell, thus enabling efficient reprogramming. The kinetics of this process were faster than that of primary fibroblasts but similar to keratinocytes, with the highest efficiency occurring after 16 days of factor expression. In vitro-derived fibroblasts appeared to be more amenable to reprogramming than primary fibroblasts as previously observed (Yu et al, 2007; Park et al, Nature), which may reflect a lack of exposure to potent differentiation cues in vitro. Without wishing to be bound by theory, four lines of evidence indicate that the reprogramming of hiPS-derived differentiated cells is representative of the process that occurs in primary cells: 1) hiPS-derived fibroblast-like cells lacked detectable expression of key pluripotency genes, 2) no colonies formed in the absence of doxycycline, 3) clones that did not reactivate all factors could not successfully form secondary hiPS cells, and 4) visual tracking of colonies demonstrated that cells transit through a non-hES-like state prior to becoming bona fide hiPS colonies. These data collectively support the use of a secondary system as a platform for mechanistic dissection of the reprogramming process.

Despite the fact that viral transgenes were reactivated in most of the hiPS-derived cells, the frequency of reprogramming remained quite low, ranging from 1-3%. The basis for the low efficiency is poorly understood but may reflect a multitude of factors such as the starting cell type, cell cycle status and the ability to undergo replication, variability in factor reactivation, and other stochastic events. Epigenetic events have been implicated in the reprogramming process; for example, it has recently been shown that valproic acid, which acts primarily as a histone deacetylase inhibitor, can enhance the efficiency of reprogramming (Huangfu et al, 2008). Also, DNA methylation and the activation of differentiation pathways have been shown to impede the reprogramming of mouse fibroblasts (Mikkelsen et al, 2008).

The secondary system of hiPS cells presents an attractive model for which to study the molecular events that underlie the reversion of human somatic cells to a pluripotent state. By providing a homogeneous population of cells that harbors all the viral transgenes, the cultures are not subject to the background of cells that do not receive all factors, allowing proper analysis of the reprogramming process. The secondary system enables chemical and genetic screening efforts to identify key molecular constituents of reprogramming, as well as important obstacles in this process, and will ultimately lend itself as a powerful tool in the development and optimization of methods to produce hiPS cells.

### Example 2

### Experimental Procedures

### Virus Production

Vectors were constructed as previously described (Stadtfeld et al, 2008b). To generate virus, 293T cells were transfected at 30% confluence using Fugene 6 reagent (ROCHE®). For a 10 cm plate, 560 uL DMEM, 27 uL Fugene, and 12 ug DNA (4:3:2 vector: Δ8.9:vsv-g) was used. Virus was harvested over 3 days and concentrated 300-fold. For a standard infection in a 35 mm dish (˜105 cells), 10 uL rtTA+5 uL factors (OCT4, SOX2, KLF4, NANOG)+2 uL cMYC was used in an overnight infection supplemented with 6 ug/mL polybrene.

Cell Culture and hiPS Cell Generation

Fibroblasts were grown in DMEM with 10% FBS, non-essential amino acids, glutamine, and β-mercaptoethanol; keratinocytes were cultured on collagen IV in keratinocyte serum-free medium and growth supplement (INVITROGEN™). Human ES and iPS cells were cultured as previously described (Cowan et al, NEJM, 2004). hiPS cells were generated as follows: Day 0, to μg/mL dox in hES media+2% defined FBS (Gibco) for fibroblasts, 1% FBS for keratinocytes; Day 2, hES+1 μg/mL dox+1% FBS (fibroblasts) or hES+dox (keratinocytes); Day 4, hES+1 μg/mL dox; Day 10, hES+0.5 μg/mL dox (continued until colonies appeared).

### Differentiation

For in vitro differentiation, hiPS cell colonies were mechanically picked and placed in suspension culture with fibroblast media. After one week, embryoid bodies were plated to adherent conditions with gelatin. For teratoma formation, ˜10⁷ hiPS cells were pelleted and injected into SCID mice, either subcutaneously above the dorsal flank or underneath the kidney capsule. Tumors were harvested after 10-12 weeks and processed for histological analysis.

### Bisulfite Sequencing

Genomic DNA was converted using the Epitect bisulfite kit (QIAGEN®). OCT4 and NANOG promoters were PCR amplified using primers listed in Table 2. PCR products were cloned into TOPO vectors and sequenced.

### Immunostaining

Immunostaining was performed using the following antibodies: α-Oct4 (sc-8628, Santa Cruz Biotech), α-Tra-1-81 (MAB4381, Millipore), α-β-III Tubulin (T2200, Sigma), α-cardiac troponin T (clone 13-11, Neomarkers), α-myosin heavy chain (clone MF20, Developmental Studies Hybridoma Bank), α-AFP (sc-15375, Santa Cruz Biotech).

qPCR

RNA was extracted using a QIAGEN RNEASY® kit, then converted to cDNA with the SUPERSCRIPT III FIRST-STRAND™ synthesis system (INVITROGEN™) using oligo-dT primers. qRT-PCRs were carried out using BRILLIANT II SYBR GREEN™ mix (STRATAGENE™) and run on a STRATAGENE™ MXPro400. Reactions were carried out in duplicate with −RT controls, and data was analyzed using the delta-delta Ct method. Primer sequences are listed in Table 2.

### Whole Genome Expression Analysis

Total RNA was isolated using an RNeasy kit (QIAGEN®). Samples were processed as independent triplicates. RNA probes for microarray hybridization were prepared and hybridized to Affymetrix HGU133 plus 2 oligonucleotide microarrays. Data was extracted and analyzed using Rosetta Resolver system. During importation, the data was subjected to background correction, intrachip normalization, and the Rosetta Resolver Affymetrix GeneChip error model (Weng et al, 2006). For the generation of intensity plots, genes that showed greater than a two-fold difference in expression value (p<0.01) in HUES8 hES cells and BJ fibroblasts were noted (19,663 probes) and their expression analyzed. A hierarchical clustering was performed.

All references described herein are incorporated in their entirety herein by reference.

### REFERENCES

1. Aoi T, Yae K, Nakagawa M, Ichisaka T, Okita K, Takahashi K, Chiba T, Yamanaka S. (2008). Science. [DOI: 10.1126/science.1154884]
2. Hanna, J., Markoulaki, S., Schorderet, P., Carey, B. W., Beard, C., Wernig, M., Creyghton, M. P., Steine, E. J., Cassady, J. P., Foreman, R., et al. (2008). Cell 133, 250-264.
3. Huangfu, D., Maehr, R., Guo, W., Eijkelenboom, A., Snitow, M., Chen, A. E., Melton D. A. (2008). Nat Biotechnol 26, 795-797.
4. Kopp, J. L., Ormsbee, B. D., Desler, M., and Rizzino, A. (2008). Stem Cells 26, 903-911.
5. Lowry, W. E., Richter, L., Yachechko, R., Pyle, A. D., Tchieu, J., Sridharan, R., Clark, A. T., and Plath, K. (2008). Proc Natl Acad Sci USA 105, 2883-2888.
6. Maherali, N., Sridharan, R., Xie, W., Utikal, J., Eminli, S., Arnold, K., Stadtfeld, M., Yachechko, R., Tchieu, J., Jaenisch, R., et al. (2007). Cell Stem Cell 1, 55-70.
7. Mikkelsen, T. S., Hanna, J., Zhang, X., Ku, M., Wernig, M., Schorderet, P., Bernstein, B. E., Jaenisch, R., Lander, E. S., and Meissner, A. (2008). Nature 454, 49-55.
8. Niwa, H., Miyazaki, J., and Smith, A. G. (2000). Nat Genet 24, 372-376.
9. Okita, K., Ichisaka, T., and Yamanaka, S. (2007). Nature 448, 313-317.
10. Park, I. H., Zhao, R., West, J. A., Yabuuchi, A., Huo, H., Ince, T. A., Lerou, P. H., Lensch, M. W., and Daley, G. Q. (2008). Nature 451, 141-146.
11. Stadtfeld, M., Maherali, N., Breault, D. T., and Hochedlinger, K. (2008a). Cell Stem Cell, 2, 230-240.
12. Stadtfeld, M., Brennand, K., and Hochedlinger, K. (2008b). Curr Biol 18, 890-894.
13. Takahashi, K., and Yamanaka, S. (2006). Cell 126, 663-676.
14. Takahashi, K., Tanabe, K., Ohnuki, M., Narita, M., Ichisaka, T., Tomoda, K., and Yamanaka, S. (2007). Cell 131, 861-872.
15. Weng, L., Dai, H., Zhan, Y., He, Y., Stepaniants, S. B., and Bassett, D. E. (2006). Bioinformatics 22, 1111-1121.
16. Wernig, M., Lengner, C. J., Hanna, J., Lodato, M. A., Steine, E. J., Foreman, R., Staerk, J., Markoulaki, S., and Jaenisch, R. (2008). Nat Biotechnol AOP.
17. Wernig, M., Meissner, A., Foreman, R., Brambrink, T., Ku, M., Hochedlinger, K., Bernstein, B. E., and Jaenisch, R. (2007). Nature 448, 318-324.
18. Yang, J., Weinberg, R. A. (2008). Dev Cell 14, 818-29.
19. Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., et al. (2007). Science 318, 1917-1920.

TABLE 1

| | | | Current passage | |
|---|---|---|---|---|
| Parental cell | Factors | Clones derived | (July 2008) | Karyotype |
| BJ fibroblasts (neonatal | 4 + GFP | #5 | 30 | 46, XY |
| foreskin) | | #6 | 20 | 47, XY, +mar [2] |
| | | | | 48, XY, +5, +13 [1] |
| | | | | 46, XY[17] |
| | | #8 | 35 | 46, XY |
| | 4 + NANOG | #11 | 28 | 46, XY |
| | | #12 | 30 | 46, XY |
| BJ hiPS #12-derived | (4 + NANOG) | #12 secondary | 20 | 46, XY |
| fibroblast-like cells | | | | |
| Epidermal keratinocytes | 4 + NANOG | #1 | 7 | ND |
| (neonatal foreskin) | | #2 | 5 | ND |
| | | #4 | 6 | ND |

4 factors = OCT4, SOX2, cMYC, KLF4
ND = not determined

TABLE 2

| | | |
|---|---|---|
| Gene name | Forward primer sequence | Reverse primer sequence |

| | | |
|---|---|---|
| Total OCT4 | GAG GAG TCC CAG GAC ATC AA | TGG CTG AAT ACC TTC CCA AA |
| Total SOX2 | AGC TAC AGC ATG ATG CAG GA | GGT CAT GGA GTT GTA CTG CA |
| Total cMYC | ACT CTG AGG AGG AAC AAG AA | TGG AGA CGT GGC ACC TCT T |
| Total KLF4 | CCC AAT TAC CCA TCC TTC CT | ACG ATC GTC TTC CCC TCT TT |
| Total NANOG | TAC CTC AGC CTC CAG CAG AT | CCT TCT GCG TCA CAC CAT T |
| Lentiviral OCT4 | CCC CTG TCT CTG TCA CCA CT | CCA CAT AGC GTA AAA GGA GCA |
| Lentiviral SOX2 | ACA CTG CCC CTC TCA CAC AT | CAT AGC GTA AAA GGA GCA ACA |
| Lentiviral cMYC | AAG AGG ACT TGT TGC GGA AA | TTG TAA TCC AGA GGT TGA TTA TCG |
| Lentiviral KLF4 | GAC CAC CTC GCC TTA CAC AT | CAT AGC GTA AAA GGA GCA ACA |
| Lentiviral NANOG | ACA TGC AAC CTG AAG ACG TG | CAC ATA GCG TAA AAG GAG CAA |
| Endogenous OCT4 | TGT ACT CCT CGG TCC CTT TC | TCC AGG TTT TCT TTC CCT AGC |
| Endogenous SOX2 | GCT AGT CTC CAA GCG ACG AA | GCA AGA AGC CTC TCC TTG AA |
| Endogenous cMYC | CGG AAC TCT TGT GCG TAA GG | CTC AGC CAA GGT TGT GAG GT |
| Endogenous KLF4 | TAT GAC CCA CAC TGC CAG AA | TGG GAA CTT GAC CAT GAT TG |
| Endogenous NANOG | CAG TCT GGA CAC TGG CTG AA | CTC GCT GAT TAG GCT CCA AC |
| OCT4 promoter | AAG TTT TTG TGG GGG ATT TGT AT | CCA CCC ACT AAC CTT AAC CTC TA |
| NANOG promoter | TTA ATT TAT TGG GAT TAT AGG GGT G | AAA CCT AAA AAC AAA CCC AAC AAC |
| DNMT3B | CCA ATC CTG GAG GCT ATC CG | ACT GGG GTG TCA GAG CCA T |
| TDGF1 (CRIPTO) | AAG ATG GCC CGC TTC TCT TAC | AGA TGG ACG AGC AAA TTC CTG |
| ZFP42 (REX1) | AAC GGG CAA AGA CAA GAC AC | GCT GAC AGG TTC TAT TTC CGC |
| GAPDH | TGT TGC CAT CAA TGA CCC CTT | CTC CAC GAC GTA CTC AGC G |

1. A method for producing an induced pluripotent stem cell from a somatic cell, the method comprising:
(a) contacting a somatic cell with an inducible lentiviral vector comprising a nucleic acid sequence encoding at least one reprogramming factor under the control of an inducible expression control element;
(b) placing said somatic cell of step (a) under conditions that induce expression via inducible expression control element; and
(c) isolating a reprogrammed cell of step (b).

2. The method of claim 1, wherein step (b) comprises contacting said somatic cell with an effective amount of a regulatory agent that controls expression from said inducible expression from said inducible expression control element.

3. The method of claim 1, wherein said inducible expression control element is a tetracycline responsive element.

4. The method of claim 1, wherein step (b) comprises contacting said somatic cell of step (a) with an effective amount of doxycycline.

5. The method of claim 4, wherein withdrawal of said effective amount of a regulatory agent permits differentiation of said reprogrammed cell.

6. The method of claim 1, wherein said somatic cell comprises a human cell.

7. The method of claim 1, wherein said somatic cell comprises a fibroblast.

8. The method of claim 1, wherein said somatic cell comprises a keratinocyte.

9. The method of claim 1, wherein said reprogramming factor is selected from the group consisting of Oct4, Sox2, c-Myc and Klf4.

10. The method of claim 1, wherein said reprogramming factor includes each of Oct4, Sox2, c-Myc, and Klf4.

11. The method of claim 10, wherein said reprogramming factor further comprises NANOG.

12. The method of claim 1, wherein production of said induced pluripotent stem cell is evidenced by detection of a stem cell marker.

13. The method of claim 12, wherein said stem cell marker is selected from the group consisting of SSEA1, CD9, Nanog, Fbx15, Ecat1, Esg1, Eras, Gdf3, Fgf4, Cripto, Dax1, Zpf296, S1c2a3, Rex1, Utf1, Oct4, SOX2, and Nat1.

14. A secondary cell composition comprising a cell having a nucleic acid encoding at least one reprogramming factor operatively linked to an inducible promoter, wherein said at least one reprogramming factor is not substantially expressed.

15. The composition of claim 14, wherein said cell comprises a fibroblast phenotype.

16. The composition of claim 14, wherein induction of expression of said at least one reprogramming factor initiates cellular reprogramming to a iPS cell phenotype.

17. The composition of claim 14, wherein said cell comprises a human cell.

18. The composition of claim 11, wherein said reprogramming factor is selected from the group consisting of Oct4, Sox2, c-Myc, NANOG, and Klf4.

19. The composition of claim 14, wherein said reprogramming factor includes each of Oct4, Sox2, c-Myc, and Klf4.

20. The composition of claim 19, wherein said reprogramming factor further comprises NANOG.

21. The composition of claim 15, wherein said cell is propagated in culture.

22. The composition of claim 14, wherein said reprogramming factor operatively linked to an inducible promoter is integrated into the genome of said cell.

23. The composition of claim 22, wherein more than one copy of said reprogramming factor is present in each cell.


SEQUENCE LISTING (42 sequences)

SEQ ID NO: 1 - DNA, 20 bp, Artificial Sequence
  Description of Artificial Sequence Synthetic primer
gaggagtcccaggacatcaa

SEQ ID NO: 2 - DNA, 20 bp, Artificial Sequence
  Description of Artificial Sequence Synthetic primer
agctacagcatgatgcagga

SEQ ID NO: 3 - DNA, 20 bp, Artificial Sequence
  Description of Artificial Sequence Synthetic primer
actctgaggaggaacaagaa

SEQ ID NO: 4 - DNA, 20 bp, Artificial Sequence
  Description of Artificial Sequence Synthetic primer
cccaattacccatccttcct

SEQ ID NO: 5 - DNA, 20 bp, Artificial Sequence
  Description of Artificial Sequence Synthetic primer
tacctcagcctccagcagat

SEQ ID NO: 6 - DNA, 20 bp, Artificial Sequence
  Description of Artificial Sequence Synthetic primer
cccctgtctctgtcaccact

SEQ ID NO: 7 - DNA, 20 bp, Artificial Sequence
  Description of Artificial Sequence Synthetic primer
acactgcccctctcacacat

SEQ ID NO: 8 - DNA, 20 bp, Artificial Sequence
  Description of Artificial Sequence Synthetic primer
aagaggacttgttgcggaaa

SEQ ID NO: 9 - DNA, 20 bp, Artificial Sequence
  Description of Artificial Sequence Synthetic primer
gaccacctcgccttacacat

SEQ ID NO: 10 - DNA, 20 bp, Artificial Sequence
  Description of Artificial Sequence Synthetic primer
acatgcaacctgaagacgtg

SEQ ID NO: 11 - DNA, 20 bp, Artificial Sequence
  Description of Artificial Sequence Synthetic primer
tgtactcctcggtccctttc

SEQ ID NO: 12 - DNA, 20 bp, Artificial Sequence
  Description of Artificial Sequence Synthetic primer
gctagtctccaagcgacgaa

SEQ ID NO: 13 - DNA, 20 bp, Artificial Sequence
  Description of Artificial Sequence Synthetic primer
cggaactcttgtgcgtaagg

SEQ ID NO: 14 - DNA, 20 bp, Artificial Sequence
  Description of Artificial Sequence Synthetic primer
tatgacccacactgccagaa

SEQ ID NO: 15 - DNA, 20 bp, Artificial Sequence
  Description of Artificial Sequence Synthetic primer
cagtctggacactggctgaa

SEQ ID NO: 16 - DNA, 23 bp, Artificial Sequence
  Description of Artificial Sequence Synthetic primer
aagtttttgtgggggatttgtat

SEQ ID NO: 17 - DNA, 25 bp, Artificial Sequence
  Description of Artificial Sequence Synthetic primer
ttaatttattgggattataggggtg

SEQ ID NO: 18 - DNA, 20 bp, Artificial Sequence
  Description of Artificial Sequence Synthetic primer
ccaatcctggaggctatccg

SEQ ID NO: 19 - DNA, 21 bp, Artificial Sequence
  Description of Artificial Sequence Synthetic primer
aagatggcccgcttctcttac

SEQ ID NO: 20 - DNA, 20 bp, Artificial Sequence
  Description of Artificial Sequence Synthetic primer
aacgggcaaagacaagacac

SEQ ID NO: 21 - DNA, 21 bp, Artificial Sequence
  Description of Artificial Sequence Synthetic primer
tgttgccatcaatgacccctt

SEQ ID NO: 22 - DNA, 20 bp, Artificial Sequence
  Description of Artificial Sequence Synthetic primer
tggctgaataccttcccaaa

SEQ ID NO: 23 - DNA, 20 bp, Artificial Sequence
  Description of Artificial Sequence Synthetic primer
ggtcatggagttgtactgca

SEQ ID NO: 24 - DNA, 19 bp, Artificial Sequence
  Description of Artificial Sequence Synthetic primer
tggagacgtggcacctctt

SEQ ID NO: 25 - DNA, 20 bp, Artificial Sequence
  Description of Artificial Sequence Synthetic primer
acgatcgtcttcccctcttt

SEQ ID NO: 26 - DNA, 19 bp, Artificial Sequence
  Description of Artificial Sequence Synthetic primer
ccttctgcgtcacaccatt

SEQ ID NO: 27 - DNA, 21 bp, Artificial Sequence
  Description of Artificial Sequence Synthetic primer
ccacatagcgtaaaaggagca

SEQ ID NO: 28 - DNA, 21 bp, Artificial Sequence
  Description of Artificial Sequence Synthetic primer
catagcgtaaaaggagcaaca

SEQ ID NO: 29 - DNA, 24 bp, Artificial Sequence
  Description of Artificial Sequence Synthetic primer
ttgtaatccagaggttgattatcg

SEQ ID NO: 30 - DNA, 21 bp, Artificial Sequence
  Description of Artificial Sequence Synthetic primer
catagcgtaaaaggagcaaca

SEQ ID NO: 31 - DNA, 21 bp, Artificial Sequence
  Description of Artificial Sequence Synthetic primer
cacatagcgtaaaaggagcaa

SEQ ID NO: 32 - DNA, 21 bp, Artificial Sequence
  Description of Artificial Sequence Synthetic primer
tccaggttttctttccctagc

SEQ ID NO: 33 - DNA, 20 bp, Artificial Sequence
  Description of Artificial Sequence Synthetic primer
gcaagaagcctctccttgaa

SEQ ID NO: 34 - DNA, 20 bp, Artificial Sequence
  Description of Artificial Sequence Synthetic primer
ctcagccaaggttgtgaggt

SEQ ID NO: 35 - DNA, 20 bp, Artificial Sequence
  Description of Artificial Sequence Synthetic primer
tgggaacttgaccatgattg

SEQ ID NO: 36 - DNA, 20 bp, Artificial Sequence
  Description of Artificial Sequence Synthetic primer
ctcgctgattaggctccaac

SEQ ID NO: 37 - DNA, 23 bp, Artificial Sequence
  Description of Artificial Sequence Synthetic primer
ccacccactaaccttaacctcta

SEQ ID NO: 38 - DNA, 24 bp, Artificial Sequence
  Description of Artificial Sequence Synthetic primer
aaacctaaaaacaaacccaacaac

SEQ ID NO: 39 - DNA, 19 bp, Artificial Sequence
  Description of Artificial Sequence Synthetic primer
actggggtgtcagagccat

SEQ ID NO: 40 - DNA, 21 bp, Artificial Sequence
  Description of Artificial Sequence Synthetic primer
agatggacgagcaaattcctg

SEQ ID NO: 41 - DNA, 21 bp, Artificial Sequence
  Description of Artificial Sequence Synthetic primer
gctgacaggttctatttccgc

SEQ ID NO: 42 - DNA, 19 bp, Artificial Sequence
  Description of Artificial Sequence Synthetic primer
ctccacgacgtactcagcg